(19) United States
(12) Patent Application Publication
(10) Pub. No.: US 2022/0370091 A1
(43) Pub. Date: Nov. 24, 2022

(54) SURGICAL CUTTING BLADE USING COMPOSITE MATERIALS

(71) Applicant: Quadvantage Technology, Inc., Raleigh, NC (US)

(72) Inventors: Thomas Gutierrez, Macon, NC (US); Paul Leach Burroughs, III, Raleigh, NC (US)

(21) Appl. No.: 17/323,736

(22) Filed: May 18, 2021

(51) Int. Cl. A61B 17/3211; A61L 31/08; A61L 31/14

(52) U.S. Cl. CPC A61B 17/3211; A61L 31/084; A61L 31/14

(57) ABSTRACT

A guide is disclosed that operates to guide in the creation of a bone spur to facilitate harvesting a quadriceps tendon and includes an end having a width equal to a desired graft width with a terminal slope equal to a cutting angle desired. In use, the guide may be initially positioned parallel to the femur to form guide cuts in the patella. The guide is then rotated ninety degrees to allow an angled cut at the guide cut. The guide is again rotated, this time one hundred eighty degrees and a second angled cut made on the other guide cut. A final lateral cut is made and the bone spur may be lifted from the patella.

### FIELD OF THE DISCLOSURE

The present disclosure relates to a blade suitable for use in a surgical environment made from composite materials.

### BACKGROUND

Most people can go through the majority of their life without ever caring or knowing how complicated a structure the knee that helps them walk is. However, the knee remains a fragile mechanical structure that is readily susceptible to damage. While medical advances have made repairing the knee possible, repair of certain types of injuries results in other long-term effects. To assist the reader in appreciating the elegance of the present disclosure, FIG. 1 is provided with a brief explanation of the components of the knee.

For the purposes of the present disclosure, and as illustrated, the knee may be composed of the quadriceps muscles 100, the femur 102, the articular cartilage 104, the lateral condyle 106, the posterior cruciate ligament (PCL) 108, the anterior cruciate ligament (ACL) 110, the lateral collateral ligament 112, the fibula 114, the tibia 116, the patellar tendon 118, the meniscus 120, the medial collateral ligament (MCL) 122, the patella 124 (shown slightly displaced to the side—it normally rests in the center of the knee), and the quadriceps tendon 126. Of particular interest for the purposes of the present disclosure is the ACL 110 and what is done to repair the ACL 110.

ACL tears are common in athletes and are usually season-ending injuries. The ACL 110 cannot heal—it must be surgically reconstructed. The reconstruction requires replacement tissue. The most common tissue used is a central slip of the patient's own patellar tendon 118. In practice, the patellar tendon 118 has proven to be generally effective, but the size of the graft that can be used is limited to the size of the patient's own patellar tendon 118. As a rule of thumb, only a third of the patellar tendon 118 may be harvested as a graft. Thus, a doctor will measure the width of the patellar tendon 118, divide by three, and take the middle third of the patellar tendon 118. Such harvested grafts are rarely more than ten millimeters (10 mm) wide and may be smaller. Taking this tissue from a person's patellar tendon 118 also causes significant pain and discomfort in the post-operative healing period, which may last up to a year, and up to twenty (20) percent of these patients are left with chronic anterior knee pain.

Some doctors recommend and use other graft sources, such as cadaver grafts, but cadaver grafts have a higher failure rate. Additionally, there is a non-zero chance of disease transmission or rejection by the patient's immune system. As a final drawback, cadaver grafts are usually quite expensive and may not be covered by some insurance companies.

Other doctors use hamstring tendons (e.g., the distal semitendinosus tendon) because the scar created during harvesting is relatively small and there is less pain during the rehabilitation, but again, the hamstring tendon has its own collection of disadvantages. The disadvantages include the fact that once the graft is taken, a patient's hamstring will never recover to its previous strength. Further, all hamstring reconstructions stretch and are looser than the original ACL 110. This loosening is particularly problematic in younger female athletes.

Another alternative graft source is the quadriceps tendon 126. The quadriceps tendon 126 is larger and stronger than either the patellar tendon 118 or the hamstring tendon. The quadriceps tendon 126 is likewise stiffer and less prone to stretching or plastic deformation. However, the qualities that make the quadriceps tendon 126 attractive also contribute to the difficulty in harvesting a graft from the quadriceps tendon 126. Existing surgical implements require a large incision up the longitudinal axis of the femur 102 on the front or ventral/anterior side of the thigh to cut down to the level of the quadriceps tendon 126, resulting in a large post-operative scar. Additionally, the quadriceps tendon 126 has a consistency similar to the proverbial shoe leather, making it difficult to cut. However, an ACL 110 repaired with grafts from the quadriceps tendon 126 generally result in almost no anterior knee pain postoperatively over the short or long term and patients recover quicker.

U.S. Pat. Nos. 8,894,672; 8,894,675; 8,894,676; 9,044,260; 9,107,700; and 9,474,535 provide a number of devices designed to create a graft from the quadriceps tendon 126 well as a number of secondary cutting implements to trim the distal end of the graft. While these cutting implements are adequate to perform their intended purpose, there remains room for improvement.

### SUMMARY

The present disclosure provides a surgical cutting blade using composite materials. In an exemplary aspect, the blade may be circularly shaped with a triangular cut-out. The cut-out allows the overall diameter of the circularly-shaped blade to remain appropriately sized for tendon harvesting (e.g., around 10 millimeters (mm)), while still allowing a similarly-sized equilateral triangle piece of patella to pass through the blade. In a second exemplary aspect, the reusability and sharpness of the surgical cutting blade may be improved by including a first material over which a diamond-like coating is applied. The first material may be steel, ceramic, or another glass-like material such as sapphire or tetragonal zirconia. Such a composite material blade can retain a sharp edge through multiple sterilization processes. It should be appreciated that blades from these materials may not be limited to the blade with a cut-out.

In this regard, in one aspect, a surgical blade is disclosed. The surgical blade comprises a base material having a sharpened tip configured to cut. The surgical blade also comprises a coating comprising a diamond-like material.

In another aspect, a surgical cutting tool is disclosed. The surgical cutting tool comprises a handle. The handle comprises a body having a first end and a second end. The handle also comprises threads disposed proximate the second end. The surgical cutting tool also comprises a blade. The blade comprises a blade body comprising a first blade end and a second blade end. The blade also comprises second threads disposed proximate the first blade end, the second threads fitting complementarily with the threads of the handle. The blade also comprises a central portion disposed between the first blade end and the second blade end. The second blade end tapers inwardly from the central portion to a terminal portion having a generally circular cross-section and having an arc removed along at least 90 degrees but less than 180 degrees therefrom.

Those skilled in the art will appreciate the scope of the disclosure and realize additional aspects thereof after reading the following detailed description in association with the accompanying drawings.

### BRIEF DESCRIPTION OF THE DRAWINGS

The accompanying drawings incorporated in and forming a part of this specification illustrate several aspects of the disclosure, and together with the description serve to explain the principles of the disclosure.

FIG. 1 illustrates a conventional knee;

FIG. 2 illustrates a flowchart describing the process of harvesting a quadriceps tendon;

FIG. 3 illustrates an exploded view of a quadriceps tendon cutter with a detachable threaded circular blade;

FIG. 4 illustrates a patellar plug that may be created as part of a quadriceps tendon harvesting operation;

FIG. 5 is an equilateral triangle corresponding to an ideal cross-section of the patellar plug shown in FIG. 4;

FIG. 6A illustrates attempting to place the patellar plug of FIG. 4 through a circular cutting blade having a diameter equal to the base of the patellar plug;

FIG. 6B illustrates placing the patellar plug of FIG. 4 through a circular cutting blade sized to accommodate the base and height of the patellar plug;

FIG. 7A is a side view of a surgical cutting blade with a cut-out to accommodate the patellar plug according to an exemplary aspect of the present disclosure;

FIG. 7B is perspective view of the surgical cutting blade of FIG. 7A;

FIGS. 8A-8D illustrate cross-sectional views of a cutting blade made from composite materials according to an exemplary aspect of the present disclosure; and

FIGS. 9A-9D illustrate additional blades that may be made from the composite materials described in FIGS. 8A-8D.

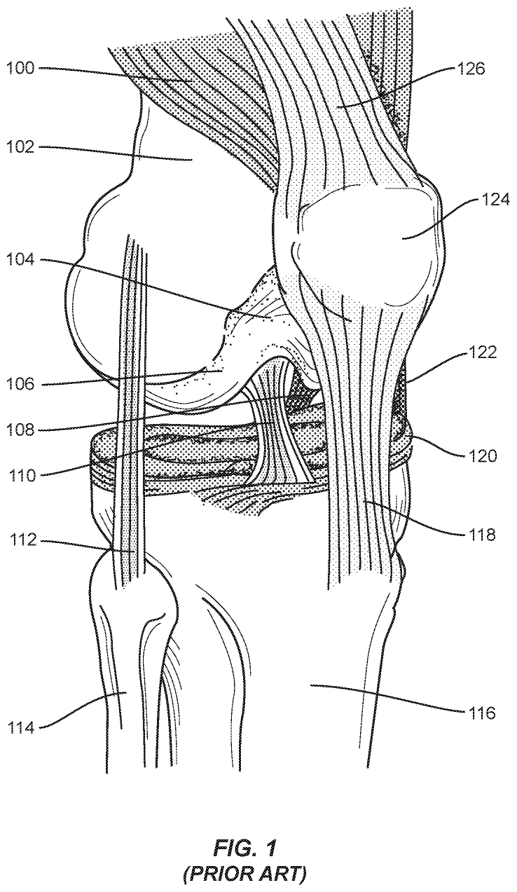

### DETAILED DESCRIPTION

The embodiments set forth below represent the necessary information to enable those skilled in the art to practice the disclosure and illustrate the best mode of practicing the disclosure. Upon reading the following description in light of the accompanying drawings, those skilled in the art will understand the concepts of the disclosure and will recognize applications of these concepts not particularly addressed herein. It should be understood that these concepts and applications fall within the scope of the disclosure and the accompanying claims.

The present disclosure provides a surgical cutting blade using composite materials. In an exemplary aspect, the blade may be circularly shaped with a triangular cut-out. The cut-out allows the overall diameter of the circularly-shaped blade to remain appropriately sized for tendon harvesting (e.g., around 10 millimeters (mm)), while still allowing a similarly-sized equilateral triangle piece of patella to pass through the blade. In a second exemplary aspect, the reusability and sharpness of the surgical cutting blade may be improved by including a first material over which a diamond-like coating is applied. The first material may be steel, ceramic, or another glass-like material such as sapphire or tetragonal zirconia. Such a composite material blade can retain a sharp edge through multiple sterilization processes. It should be appreciated that blades from these materials may not be limited to the blade with a cut-out.

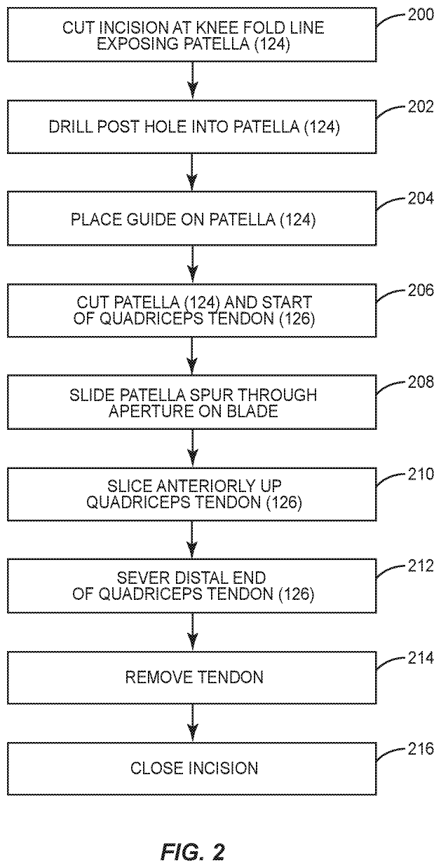

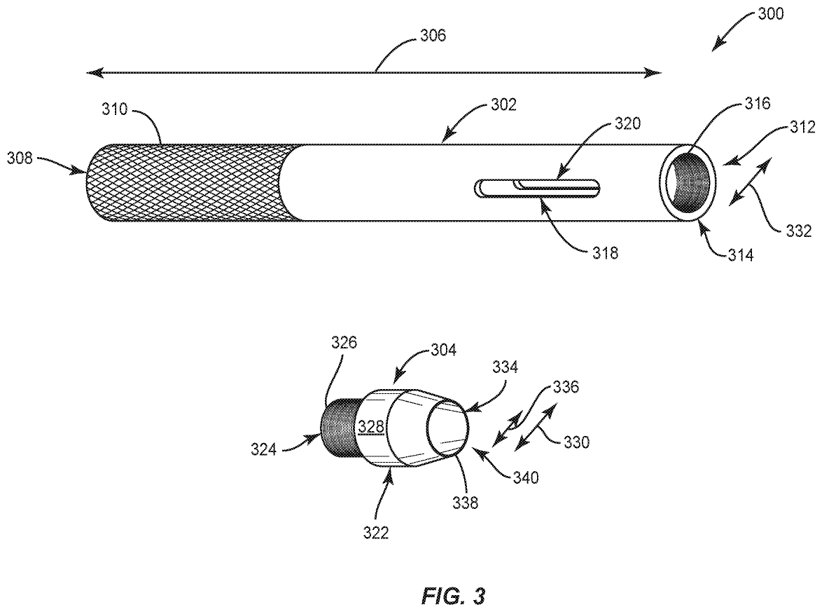

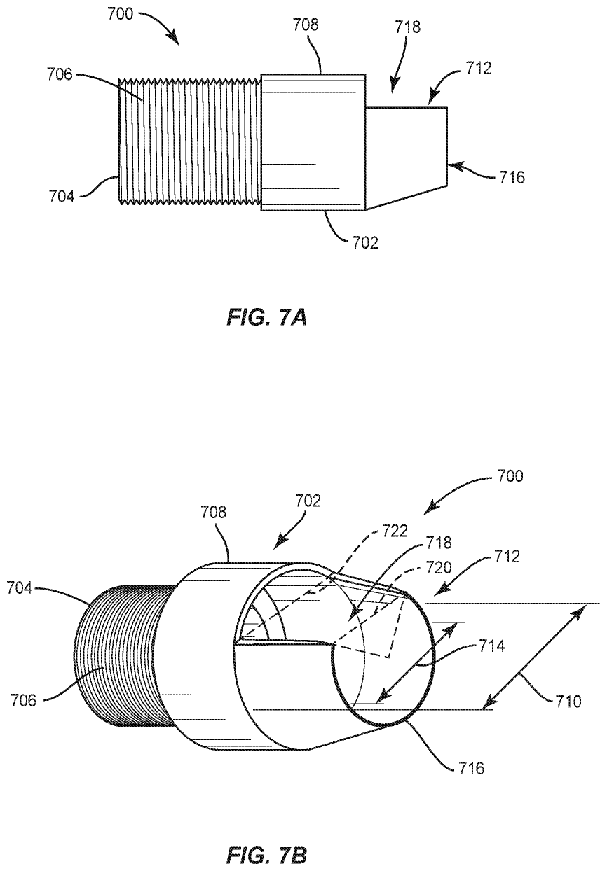

Before addressing the particular structure of the surgical blades of the present disclosure, a brief overview of the process that harvests a quadriceps tendon is provided with reference to FIG. 2. A discussion of blades according to the present disclosure begins below with reference to FIG. 3 along with a discussion of some shortcomings of a pure circular blade. A discussion of a blade capable of accommodating a bone plug without being unduly large begins below with reference to FIG. 7A.

FIG. 2 illustrates a flowchart explaining how a tendon graft is harvested. The doctor makes or cuts an initial incision at a knee fold line (block 200), such as an anterior fold line, and folds the skin back to expose a portion of the patella 124. The doctor drills a post hole into the patella 124 proximate the quadriceps tendon 126 (block 202). The hole may be approximately ten to eleven millimeters (10-11 mm) deep or sufficiently deep to pass through the patella 124. The doctor then places a guide on the patella 124, with a post of the guide positioned within the hole (block 204). The doctor will choose the width of the quadriceps tendon 126 to be harvested by measuring the size of the quadriceps tendon 126 preoperatively from magnetic resonance imaging (MRI) and comparing the images to the intraoperative observations of the tendon itself. A cross-sectional area of the patellar tendon can be calculated from the MRI, and one third of this patellar tendon area can be compared to the cross-sectional areas resulting from different quadriceps options.

Next, a bone plug or spur is created by cutting into the patella 124 using the guide to guide the cuts into the patella 124, thereby creating the bone plug and the initial cut into the quadriceps tendon 126 (block 206). Using the guide disclosed in U.S. Patent Application Publication No. 2021/0015497 (which is herein incorporated by reference), the guide directs the saw blade such that the resulting bone plug is of the same lateral dimensions as the quadriceps graft that has been chosen, allowing the bone spur to be slipped through the aperture on the tendon cutting blade (block 208). The graft may be secured by sutures based through the original hole drilled at the beginning of the procedure, facilitating passage of the bone plug. The doctor then slices anteriorly up the quadriceps tendon 126 underneath the skin of the patient (block 210). When an appropriate length of the quadriceps tendon 126 has been cut, the doctor severs the distal end of the quadriceps tendon 126 (block 212). The doctor then removes the tendon (block 214) and closes the incision (block 216).

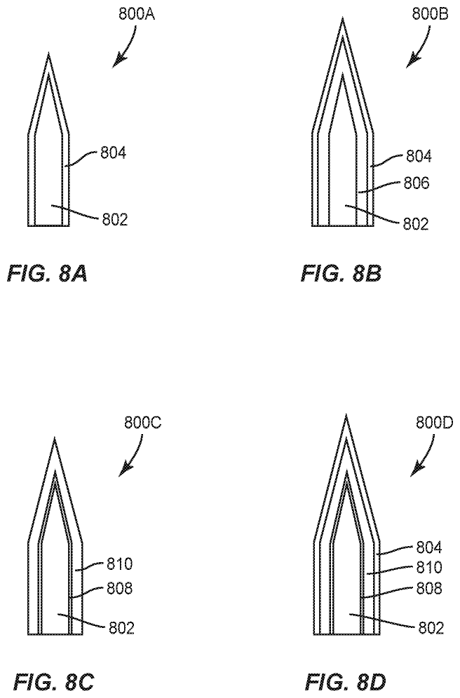

A first exemplary tendon harvesting tool 300 is illustrated in FIG. 3. Specifically, the tendon harvesting tool 300 may include a handle 302 and a blade 304. The handle 302 may be made from surgical steel or the like and is designed for reuse and as such, should be able to withstand at least two hundred (200) sterilization procedures (e.g., autoclave or the like). Additional materials are discussed below with respect to FIGS. 8A-8D. The handle 302 may be a cylinder having a longitudinal axis 306. At a first end 308 of the handle 302, a knurl pattern 310 may be provided. The knurl pattern 310 may extend approximately one third to one half the length of the handle 302 and may, for example, extend approximately four-five inches (4″-5″) along the longitudinal axis 306 from the first end 308. The cylinder may be hollow and delimit an interior space 312 therein. A second end 314 opposite the first end 308 may include interior threads 316. A slot 318 proximate the second end 314 may allow a surgeon to see a tendon graft passing into and through the handle 302 as the graft is harvested. In an exemplary aspect, the slot 318 may extend approximately two inches (2″) along the longitudinal axis 306. Further, a second slot 320 may be provided, opposite the slot 318 and sized identically. While not shown, measuring indicia (e.g., in inches, centimeters, or millimeters) may be provided on an exterior surface of the handle 302 to indicate how much of graft has been cut.

With continuing reference to FIG. 3, the blade 304 may be formed from a body 322, which has a first blade end 324, which has threads 326 proximate thereto. The threads 326 are sized and configured to mate complementarily with the threads 316 of the handle 302. The body 322 may have a central portion 328 that has an exterior diameter 330 approximately equal to an exterior diameter 332 of the handle 302. The body 322 may have a second blade end 334, which tapers from the central portion 328 to a second diameter 336. In an exemplary aspect, the second diameter 336 is between 8-12 mm. A terminal portion 338 of the second blade end 334 is circularly shaped and sharpened to provide a cutting surface capable of cutting a quadriceps tendon. The circular nature of the terminal portion 338 delimits an aperture 340.

While not shown, it should be appreciated that by providing a threaded, detachable blade 304, the blade portion of the cutting tool 300 may be swapped for a differently-sized blade portion. Thus, for one surgery, the surgeon may select a blade 304 having a diameter 336 of 8 mm and for a second, subsequent surgery, the surgeon may select a blade 304 having a diameter 336 of 11 mm. Other sizes may be used, but the most frequent graft dimensions are between 8 and 12 mm and it is expected that most blades will fall within that range. The handle 302 may be used in the first surgery, sterilized in an autoclave, and then used again with the second blade portion for the subsequent surgery.

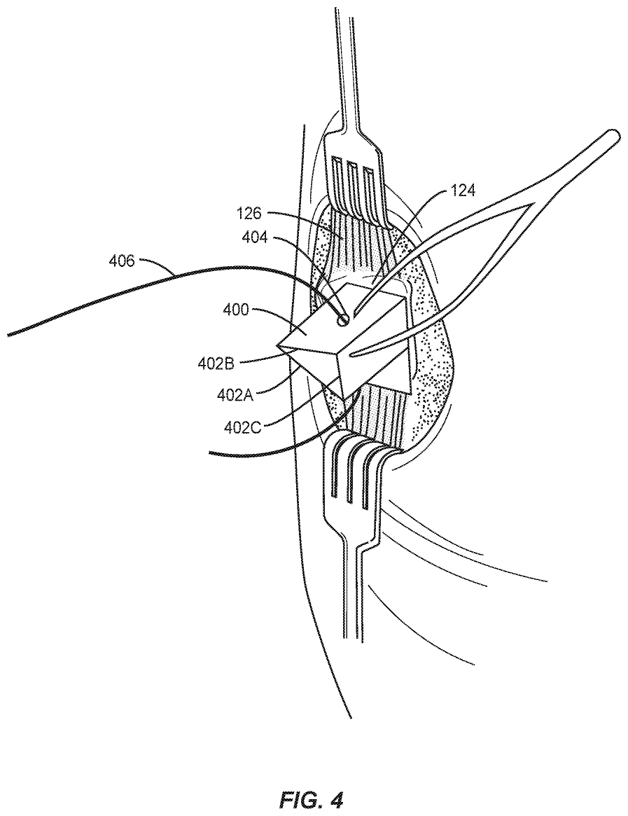

While the circular shape of the terminal portion 338 is well suited for cutting the quadriceps tendon 126, there may be an issue relative to the patella spur created in step 206 discussed above. A cut patella spur 400 is illustrated in FIG. 4 before insertion into a cutting tool 300. Specifically, a patella 124 has been cut using, for example, the guide tool from the previously discussed '497 publication. While it is possible to harvest a graft without a patella spur 400, such grafts are less user friendly in that they make attachment in the recipient more challenging. By harvesting the patella spur 400, a screw or other ready mechanical device may be used to secure the graft in the desired location. The resulting patella spur 400 has, by design, an equilateral triangular cross section, with each cross-sectional edge being approximately equal to an intended width of graft taken from the quadriceps tendon 126. For example, if the graft is to be 10 mm wide, then each edge 402A-402C of the spur 400 is likewise approximately 10 mm long. The post hole 404 formed by the drilling of step 202 may have a bit of thread 406 (e.g., suture material) placed therethrough to assist in handling the spur 400.

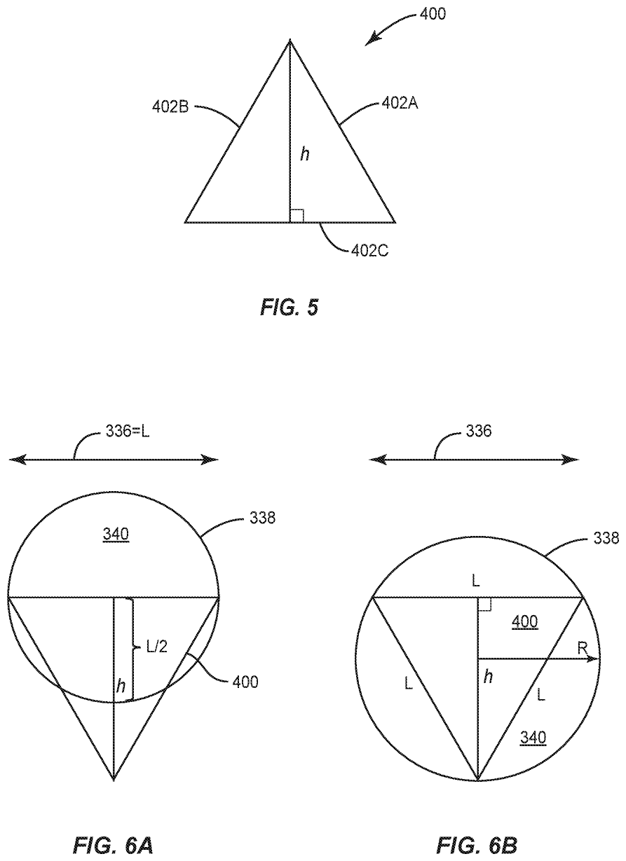

As better seen in FIG. 5, the spur 400 with edges 402A-402C each having a length L has a corresponding height h equal (by solving the Pythagorean equation) to:

$$h = \sqrt{{L}^{2} - \frac{{L}^{2}}{4}}$$

For various L of interest (e.g., 8-12 mm), h may vary from about 6.93 to about 10.39 mm. This height h becomes relevant as better illustrated in FIGS. 6A and 6B. Specifically, the terminal portion 338 with diameter 336 is illustrated in both, with a spur 400 superimposed. If, as shown in FIG. 6A, the diameter 336 is equal to L (meaning the width of the cut for the graft from the tendon 126 is equal to L), the radius is equal to L/2. Unfortunately,

$$L / 2 < \sqrt{{L}^{2} - \frac{{L}^{2}}{4}}$$

which means that the spur 400 will not readily fit through the aperture 340 formed by the terminal portion 338 if diameter 336=L. The net result of the situation illustrated in FIG. 6A is that if the surgeon selects a blade 304 having a diameter 336 equal to a desired graft width, then a bone spur 400 having the same width and being an equilateral triangle will not fit through the aperture 340.

Conversely, FIG. 6B illustrates an alternate option where the dimensions of the edges 402A-402C of the spur 400 cut perfect chords in the circle formed by the aperture 340 (i.e., the circle formed by the terminal portion 338 circumscribes the triangle of the spur 400). The diameter 338 is thus equal to:

$$2 R = \frac{2 L \sqrt{3}}{3} ,$$

where R is the radius of the circle and 2R is the diameter 336.

In this case, the diameter 336 would (for L between 8-12 mm) range from about 9.23 mm to 13.85 mm, which for any given desired L, is about 1.15 times larger than L. This means that the widest part of any blade 304 would cut a graft 1.15 times larger than desired. While functional, such approach is perhaps wasteful in that too much tendon is harvested.

Exemplary aspects of the present disclosure provide a solution that allows the surgeon to select a cutting implement having a diameter size corresponding to a desired graft size, but that also accommodates a bone spur that has the same lateral dimension as the desired graft. Specifically, the bone spur may pass through the aperture of the cutting implement without necessitating an increase in the cutting dimension of the cutting implement as better seen in FIGS. 7A and 7B. Thus, the surgeon may harvest a smaller or larger graft as needed by varying the cutting dimension of the cutting implement and remain confident that the bone spur will still fit through the terminal portion of the cutting implement essentially independent of the size of the bone spur. That is, the surgeon may create a bone spur having a first dimension during the initial part of the surgery and then, based on characteristics of the patient, decide to harvest a graft that is larger or smaller than the bone spur by selecting the appropriately-sized cutting implement. The bone spur, essentially independent of size, should still fit through the cutting implement as better illustrated in FIGS. 7A and 7B.

In particular, a blade 700 compatible with the handle 302 is illustrated. The blade 700 may be formed from a body 702, which has a first blade end 704, which has threads 706 proximate thereto. The threads 706 are sized and configured to mate complementarily with the threads 316 of the handle 302 (FIG. 3). The body 702 may have a central portion 708 that has an exterior diameter 710 approximately equal to an exterior diameter 332 of the handle 302 (FIG. 3). The body 702 may have a second blade end 712, which tapers from the central portion 708 to a second diameter 714. In an exemplary aspect, the second diameter 714 is between 8-12 mm. A terminal portion 716 of the second blade end 712 is generally circularly shaped and sharpened to provide a cutting surface capable of cutting a quadriceps tendon. However, unlike the terminal portion 338, a one hundred twenty degree (120°) arc has been removed, changing the shape of an aperture 718 and forming an L-shaped side view (better seen in FIG. 7A). Changing the shape of the aperture 718 in this fashion means that the chord 720 formed at the terminal portion 716 is wide enough to accommodate the width of the intended graft and the chord 722 formed at the junction between the central portion 708 and the beginning of the taper is wide enough to accommodate the widest part of the bone spur 400. Further, the cutting of the graft can now be limited to the desired width defined by the small diameter 714 without the extra waste shown in FIG. 6B. However, the removable and interchangeable nature of multiple blades 700 means that the surgeon can select a blade 700 sized as desired while still accommodating various sizes of bone spurs and various graft widths.

While 120 degrees is specifically contemplated, other arcs may be used ranging from more than 90 degrees to less than 180 degrees.

While a blade 304 or 700 made of surgical steel may be adequate for harvesting a quadriceps tendon, the tough nature of the quadriceps tendon will likely dull a surgical steel blade to the point where it is impractical to reuse the blade even though the surgical steel is amenable to multiple sterilizations. The result of this dulling is that the blade 304 or 700 is likely to be a single-use item, resulting in increased expense as more items are used. Further, the disposable nature of such items may lead to increased landfill use. However, exemplary aspects of the present disclosure also provide a solution to this concern. Specifically, the blade 304 and the blade 700 may be made from a composite material. In its simplest form, the composite material may be a diamond-like coating (such as that sold by UNITED PROTECTIVE TECHNOLOGIES (UPT) of 142 Cara Court, Locust NC 28097 under the tradename SPEC™ Coatings) applied to a base material. The base material may be surgical steel, a glass like material such as sapphire or tetragonal zirconia, or a ceramic material. The addition of the coating preserves the edge of the underlying material and/or makes the composite material more amenable to repeated sterilization procedures.

Thus, as illustrated in FIGS. 8A-8D, blades 800A-800D have different coatings. The blade 800A has a base material 802 and a coating 804 of diamond-like material. The blade 800B has a base material 802, a first layer 806 of silicon, and a second coating 804 of diamond-like material. The blade 800C has a base ceramic material 808 and a second layer 810 made of a-C:H:W. The blade 800D has a base ceramic material 808, a second layer 810 and a coating 804 of diamond-like material.

In general, the base materials are typically crystalline structures (stainless steel, sapphire, and Zirconia are all crystalline materials) that are susceptible, in varying degrees to corrosion, pitting, erosion, staining, and chipping. Generally, these materials can be honed to very sharp edges, however, the edges (which are key to surgical applications) quickly deteriorate in use—for example, surgical blades made from stainless steel typically become dull after several cuts. Sapphire tends to chip along the sharp edge due to stresses in its crystalline structure created during manufacturing—making the blades unusable and creating a danger that the chips will become implanted in the patient as they break off. Tetragonal Zirconia suffers from “low” temperature degradation that can cause it to crumble when subjected to high humidity and temperature such as that used to sterilize instruments in surgical settings.

To address the shortcomings of the base material, a very thin “film” is provided that protects the blade and other portions of the base material in a manner that reduces or prevents chipping, staining, pitting, corrosion, etc., thereby increasing the life of the instrument/blade, reducing the potential for accidental implantation of chipped off materials, and improving the survivability of the instrument/blade through many autoclave cycles. The coating in most cases will have a hardness/composition that is more pliable (plastic) in nature than the substrate but that has a structure that does not suffer the issues noted above that crystalline base materials have. As noted above, increasing the reusability of the device also reduces landfill waste as it lowers the number of instruments/blades disposed of by a surgical center.

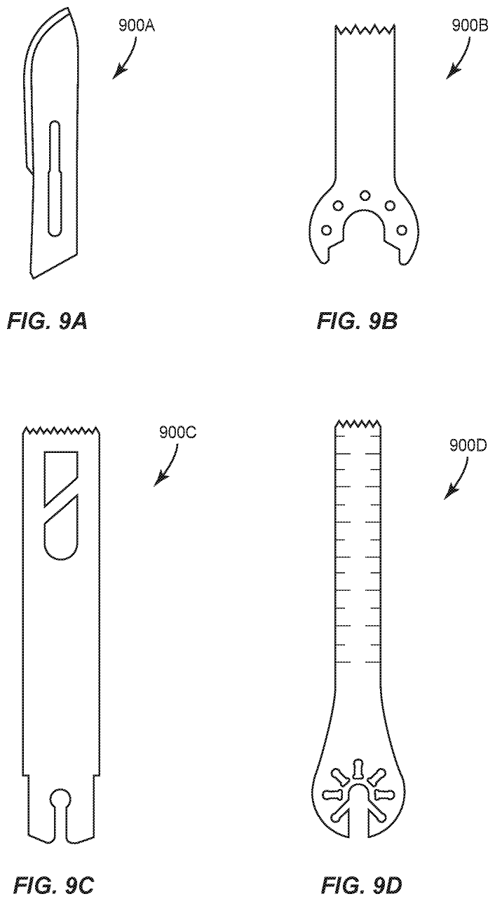

Note further that blades made from any of these composite materials do not have to be limited to the shapes of blades 304 or 700. Rather, any surgical blade made be formed from the composite material. For example, as illustrated in FIGS. 9A-9D, a scalpel 900A, saws 900B-900D, tendon cutters such as found in U.S. Pat. Nos. 8,894,672; 8,894,675; 8,894,676; 9,044,260; 9,107,700; and 9,474,535 as well as blades shown in U.S. Patent Publication No. 2020/0390463 may all be formed from the composite materials.

What is claimed is:

1. A surgical blade comprising:
a base material having a sharpened tip configured to cut; and
a coating comprising a diamond-like material.

2. The surgical blade of claim 1 formed into a scalpel blade.

3. The surgical blade of claim 1 formed into a circular blade.

4. The surgical blade of claim 1 formed into a circular blade with an arc removed therefrom.

5. The surgical blade of claim 4, wherein the arc is 120 degrees.

6. A surgical cutting tool comprising:
a handle comprising:
a body having a first end and a second end; and
threads disposed proximate the second end; and
a blade comprising:
a blade body comprising a first blade end and a second blade end;
second threads disposed proximate the first blade end, the second threads fitting complementarily with the threads of the handle;
a central portion disposed between the first blade end and the second blade end; and
the second blade end tapering inwardly from the central portion to a terminal portion having a generally circular cross-section and having an arc removed along at least 90 degrees but less than 180 degrees therefrom.

7. The surgical cutting tool of claim 6, wherein the handle comprises surgical steel.

8. The surgical cutting tool of claim 6, wherein the terminal portion comprises a composite material.

9. The surgical cutting tool of claim 6, wherein the handle comprises a cylinder having a longitudinal axis extending between the first end and the second end.

10. The surgical cutting tool of claim 9, wherein the cylinder is hollow for at least a portion of the longitudinal axis.

11. The surgical cutting tool of claim 10, wherein the handle further comprises a slot extending parallel to the longitudinal axis and communicating to an interior portion of the hollow cylinder.

12. The surgical cutting tool of claim 6, wherein the first end comprises a knurled portion.

13. The surgical cutting tool of claim 6, wherein the terminal portion has a diameter between 8-12 millimeters (mm).

14. The surgical cutting tool of claim 6, wherein the terminal portion has a diameter of 10 millimeters (mm).

15. The surgical cutting tool of claim 6, wherein the blade is configured such that a workpiece having an edge dimension larger than a diameter of the terminal portion fits through the arc allowing cutting at a desired dimension corresponding to the diameter.
